(19) United States
(12) Patent Application Publication
(10) Pub. No.: US 2010/0047894 A1
(43) Pub. Date: Feb. 25, 2010

(54) Use of Matrix Metalloproteinases, Mutated and Not Mutated, for the Preparation of Pharmaceutical Compositions, and Mutated Metalloproteinases with Increased Stability

(75) Inventors: Ivano Bertini, Firenze (IT); Claudio Luchinat, Firenze (IT); Lucia Banci, Firenze (IT); Rebecca Del Conte, Firenze (IT); Marco Fragai, Cortona (IT)

Correspondence Address: CLARK & ELBING LLP, 101 FEDERAL STREET, BOSTON, MA, 02110, US

(73) Assignee: Protera S.R.L., Sesto Fiorentino (IT)

(21) Appl. No.: 12/063,644

(22) PCT Filed: Aug. 10, 2006
(86) PCT No.: PCT/EP06/65200

§ 371 Date: Feb. 12, 2008

(30) Foreign Application Priority Data
Aug. 12, 2005 (IT) FI2005A000178

(51) Int. Cl. C12N 9/50; C07H 21/04; C12N 15/63; C12N 5/10

(52) U.S. Cl. 435/219; 536/23.2; 435/320.1; 435/325

(57) ABSTRACT

The use of matrix metalloproteinases, mutated and not mutated, for the preparation of pharmaceutical compositions useful in the treatment of pathologies associated with an accumulation of matrix bio-polymers and/or an excess of TIMPs (Tissue Inhihitors MetalloProteinases) is described; mutated matrix metalloproteinases, in which at least an aminoacid residue in a definite position in the protein, has been mutated into a hydrophilic and/or charged aminoacidic residue, obtaining an increased stability toward autoproteolysis are also described.

### FIELD OF INVENTION

The invention relates to the field of proteins and in particular to the use of matrix metalloproteinases, mutated and not mutated, for the preparation of pharmaceutical compositions, as well as to mutated matrix metalloproteinases, as defined hereinafter, having an increased stability toward autoproteolysis in respect to the corresponding not mutated proteins.

### STATE OF ART

Matrix metalloproteinases constitute a family of more than 20 different Zn-dependent enzymes, which are responsible for the degradation of extracellular matrix. The extracellular matrix components carry out a fundamental role in the modulation of cellular environment during growth, morphogenesis and tissue reparation processes. For this reason, the matrix metalloproteinases activities is subtly regulated both at transcription and activation level, as well as by the endogenous inhibitors action such as TIMPs (Tissue Inhibitors MetalloProteinases) and α₂-macroglobuline. Changes in the delicate equilibrium which regulates the matrix metalloproteinase activities are connected with arising and development of various pathologies such as scleroderma, cardiosclerosis, nephrosclerosis, hepatic fibrosis, pulmonary fibrosis and pancreatic fibrosis. Many active principles are known as inhibitors of the enzymatic activities of matrix metalloproteinases, so they are used to prepare pharmaceutical compositions useful in the treatment of pathologies which are associated with an overexpression of metalloproteinases. On the contrary, as far as the Applicant is aware of, the matrix metalloproteinases are not directly used in the pharmaceutical field as active principles in the isolated and purified form.

In spite of that, there is a huge request of these proteins both in academic and industrial fields, due to their relevance in physiological and pathological fields. Indeed, the availability of purified matrix metalloproteinases is a question of primary importance in the study of biological processes in which these proteins are involved, and in the development of candidate inhibitors.

However, the use of matrix metalloproteinases is usually difficult, complicated and expensive, because of their tendency towards autoproteolysis and the consequent degradation of the protein itself. This behaviour is due to the fact that one or more aminoacidic sequences exposed on the protein surface, possess good affinities towards the catalytic site of the protein which, in conclusion, hydrolyse itself.

To avoid this process of autoproteolysis and degradation, the use of matrix metalloproteinases requires very narrow working conditions that often make their use in experimental conditions, very intricate and sometimes even impossible.

Their transport and preservation are very complicated too, indeed manufacturing firms recommend to preserve them at a temperature of −80° C.

Another important restriction is the low concentration at which the researcher is forced to work to avoid autoproteolysis. This is another limit to the use of matrix metalloproteinases in many experiments.

To the present day, to reduce autoproteolysis phenomenon, researchers used synthetic inhibitors or self-inhibited protein, i.e. a protein that is provided with a prodomain. Nevertheless, both strategies present many limitations and do not represent a solution for this problem. To overcome these difficulties mutants of matrix metalloproteinases have been produced, in which aminoacid Glutamate, which is present in the active site of the enzyme and is involved in catalytic mechanism, is replaced by an Alanine (Morgunova, E. et al., Science 1999, 284, 1667). Indeed the Glutamate, by the carboxylic group of the side chain, coordinates the water molecule that, during hydrolysis, is responsible of the nucleophilic attack on the peptidic carbonyl (Babine, R. E. et al., Chem. Rev. 1197, 97, 1359-1472); following to Glutamate substitution the protein loses the water molecule and it is not able to perform its catalytic action anymore. As it can be easily understood, this approach is not valid in the case in which it would be necessary to keep the catalytic activity, because it generates inactive proteins.

The need is therefore deeply felt, to develop an alternative approach able to eliminate the susceptibility of these proteins to autoproteolysis without interfering neither with their catalytic activity, nor with their affinity for substrates and natural inhibitors.

### SUMMARY OF THE INVENTION

The Applicant has surprisingly found that mutation of an aminoacid in the catalytic domain of the matrix metalloproteinases, in a specific position far from the active site, is able to increase their stability towards autoproteolysis in respect to the wild-type, not mutated protein.

Moreover, the Applicant has found that both mutated and wild-type matrix metalloproteinases can be used for the preparation of pharmaceutical compositions, and in particular of compositions useful in the treatment of pathologies to which an accumulation of matrix bio-polymers and/or an excess of TIMPs is associated, such as scleroderma, cardiosclerosis, nephrosclerosis, hepatic fibrosis, pulmonary fibrosis and pancreatic fibrosis.

Subject of the present invention is therefore the catalytic domain of human matrix metalloproteinases, characterised in that said catalytic domain is mutated so that the aminoacidic residue corresponding to phenylalanine 171 according to the numbering of the sequence Accession No. P39900 (SwissProt), is an hydrophilic and/or charged aminoacidic residue.

Further subjects of the invention are: the matrix metalloproteinases comprising as catalytic domain the above said mutated catalytic domain; the DNA sequence codifying the above said mutated matrix metalloproteinases; the recombinant vector comprising the above said DNA sequence; and the isolated cell transfected or transformed with the above said recombinant vector.

A further subject of the invention is the use of human matrix metalloproteinase and of the catalytic domains thereof, optionally mutated as said above, for the preparation of pharmaceutical composition; and the so obtained pharmaceutical compositions.

Further subject of the invention is the diagnostic kit for the diagnosis of pathologies to which an accumulation of matrix bio-polymers and/or an excess of TIMPs (Tissural Inhibitors of Matrix Metalloproteinases) is associated, comprising human matrix metalloproteinases or the catalytic domains thereof, optionally mutated as said above.

Further subject of the invention is the use of human matrix metalloproteinases mutated as said above, or of the mutated catalytic domain thereof, as reagents for the pharmacological characterisation of the matrix metalloproteinase as pharmaceutical target.

Features and advantages of the invention will be described in detail in the following description.

### BRIEF DESCRIPTION OF THE DRAWINGS

FIG. 1: SDS (Sodium Dodecyl Sulphate)/polyacrylamide gel electrophoresis of wild-type and mutated MMP3 (SEQ ID No. 3) as illustrated in Example 3.

FIG. 2: SDS/polyacrylamide gel electrophoresis of wild-type and mutated MMP10 (SEQ ID No. 10) as illustrated in Example 4.

FIG. 3: SDS/polyacrylamide gel electrophoresis of wild-type and mutated MMP13 (SEQ ID No. 10) as illustrated in Example 5.

### DETAILED DESCRIPTION OF THE INVENTION

In the present invention, by the expression “hydrophilic and/or charged aminoacidic residue” is meant, for example, an aminoacidic residue selected from the group consisting of glutamine, asparagine, aspartic acid and glutamic acid; aspartic acid is preferably meant.

In the present invention, by the expression “aminoacidic residue corresponding to the phenylalanine 171 according to the numbering of the sequence Accession No. P39900 (Swiss Prot)” is meant the aminoacidic residue in the same position of the above said phenylalanine 171 as resulting in a multiple alignment, as defined hereinafter.

For the various matrix metalloproteinases (hereinafter referred to with the abbreviation MMP) not having, as such, an hydrophilic and/or charged residue in the corresponding position as defined above, i.e. for all MMPs with the exclusion of MMP-1 (SEQ ID No. 1), MMP-8 (SEQ ID No. 5), and MMP-11 (SEQ ID No. 8), the mutation is located in the following positions, identified through the multiple alignment:
for human MMP-2 (SEQ ID No. 2) and all its isoforms determined by alternative “splicing”, the aminoacidic residue that is mutated is glycine 181 according to the numbering of the sequence Accession No. P08253 (SwissProt);
for human MMP-3 (SEQ ID No. 3) and all its isoforms determined by alternative “splicing”, the aminoacidic residue that is mutated is phenylalanine 171 according to the numbering of the sequence Accession No. P08254 (SwissProt) or Q6GRF8 (TrEMBL);
for human MMP-7 (SEQ ID No. 4) and all its isoforms determined by the alternative “splicing”, the aminoacidic residue that is mutated is serine 166 according to the numbering of the sequence Accession No. P09237 (SwissProt);
for human MMP-9 (SEQ ID No. 6) and all its isoforms determined by the alternative “splicing”, the aminoacidic residue that is mutated is glycine 178 according to the numbering of the sequence Accession No. P14780 (SwissProt);
for human MMP-10 (SEQ ID No. 7) and all its isoforms determined by the alternative “splicing”, the aminoacidic residue that is mutated is phenylalanine 170 according to the numbering of the sequence Accession No. P09238 (SwissProt) or Q53HH9 (TrEMBL);
for human MMP-12 (SEQ ID No. 9) and all its isoforms determined by the alternative “splicing”, the aminoacidic residue that is mutated is phenylalanine 171 according to the numbering of the sequence Accession No. P39900 (SwissProt);
for human MMP-13 (SEQ ID No. 10) and all its isoforms determined by the alternative “splicing”, the aminoacidic residue that is mutated is phenylalanine 175 according to the numbering of the sequence Accession No. P45452 (SwissProt) or Q7Z5M0, Q7Z5M1 and Q6NWN6 (TrEMBL);
for human MMP-14 (SEQ ID No. 11) and all its isoforms determined by the alternative “splicing”, the aminoacidic residue that is mutated is serine 189 according to the numbering of the sequence Accession No. P50281 (SwissProt) and Q6GSF3 (TrEMBL);
for human MMP-15 (SEQ ID No. 12) and all its isoforms determined by the alternative “splicing”, the aminoacidic residue that is mutated is serine 209 according to the numbering of the sequence Accession No. P51511 (SwissProt) and Q7KZY0 (TrEMBL);
for human MMP-16 (SEQ ID No. 13 and SEQ ID No. 14) and all its isoforms determined by the alternative “splicing”, the aminoacidic residue that is mutated is serine 196 according to the numbering of the sequence Accession No. P51512 (SwissProt), or Q52H48 and Q14824 (TrEMBL);
for human MMP-17 (SEQ ID No. 15) and all its isoforms determined by the alternative “splicing”, the aminoacidic residue that is mutated is glycine 200 according to the numbering of the sequence Accession No. Q9ULZ9 (SwissProt) or Q5U5M0 and Q81WC3 (TrEMBL);
for human MMP-19 (SEQ ID No. 16 and SEQ ID No. 17) and all its isoforms determined by the alternative “splicing”, the aminoacidic residue that is mutated is tyrosine 165 according to the numbering of the sequence Accession No. Q99542 (TrEMBL);
for human MMP-20 (SEQ ID No. 18) and all its isoforms determined by the alternative “splicing”, the aminoacidic residue that is mutated is serine 179 according to the numbering of the sequence Accession No. 060882 (TrEMBL);
for human MMP-21 (SEQ ID No. 19) and all its isoforms determined by the alternative “splicing”, the aminoacidic residue that is mutated is cysteine 238 according to the numbering of the sequence Accession No. Q5VZP9 and Q8N119 (TrEMBL);
for human MMP-23A (SEQ ID No. 20) and all its isoforms determined by the alternative “splicing”, the aminoacidic residue that is mutated is cysteine 152 according to the numbering of the sequence Accession No. O75900 (TrEMBL);
for human MMP-23B (SEQ ID No. 21) and all its isoforms determined by the alternative “splicing”, the aminoacidic residue that is mutated is cysteine 152 according to the numbering of the sequence Accession No. Q9UBR9 (TrEMBL);
for human MMP-24 (SEQ ID No. 22) and all its isoforms determined by the alternative “splicing”, the aminoacidic residue that is mutated is serine 232 according to the numbering of the sequence Accession No. Q9Y5R2 and Q9H440 (TrEMBL);
for human MMP-25 (SEQ ID No. 23) and all its isoforms determined by the alternative “splicing”, the aminoacidic residue that is mutated is serine 182 according to the numbering of the sequence Accession No. Q9NPA2 (TrEMBL);
for human MMP-26 (SEQ ID No. 24) and all its isoforms determined by the alternative “splicing”, the aminoacidic residue that is mutated is glycine 161 according to the numbering of the sequence Accession No. Q9NRE1 (TrEMBL);
for human MMP-27 (SEQ ID No. 25) and all its isoforms determined by the alternative “splicing”, the aminoacidic residue that is mutated is cysteine 168 according to the numbering of the sequence Accession No. Q9H306 and Q6UWK6 (TrEMBL);
for human MMP-28 (SEQ ID No. 26 and SEQ ID No. 27) and all its isoforms determined by the alternative “splicing”, the aminoacidic residue that is mutated is glycine 193 according to the numbering of the sequence Accession No. Q9H239 and Q9BUG8 (TrEMBL);
for human MMP-like 1 (SEQ ID No. 28) and all its isoforms determined by the alternative “splicing”, the aminoacidic residue that is mutated is serine 106 according to the numbering of the sequence Accession No. O43923 (TrEMBL);

For the alignment the program ClustalW (1.6) has been used with the following parameters: substitution matrix Gonnet 250, Gap open 10, Gap close −1, Gap extension −2, Gap distance 4. For a complete description of the alignment methodology see Andreini C. et al., J. of Proteome Research, 2004, 3, 21, which is herewith incorporated by reference.

As showed in the following examples, the mutated proteins according to the invention presented a much more increasing stability in respect to the corresponding wild-type, not mutated protein. This makes more efficacious the use of the present mutated proteins not only as a scientific research instrument, for instance as a pharmaceutical tool to test new drugs having the matrix metalloproteinases as pharmaceutical target, but also as a pharmaceutical active principle.

The following examples are reported to illustrate, and not to limit the invention.

### Example 1

According to the invention, the mutation of the aminoacidic residues has been obtained by specific site mutagenesis. A couple of nucleotides has been synthesised to this aim, wherein the nucleotides are constituted of 35 basis complementary to the wild type gene with the exclusion of the triplet codifying the aminoacid to be mutated in the protein. With these oligonucleotides a PCR reaction has been carried out on the plasmid containing the wild type gene at an annealing tₘ temperature reduced by at least 15-20° C. respect to tₘ of the oligonucleotides. A plasmid containing the mutated gene has been so obtained. The reaction mixture has been transformed in E. coli cells, and the plasmids contained in these cells have been isolated. The plasmid containing the mutated gene has been identified by gene sequencing.

### Example 2

Catalytic domains of MMPs, wild type and mutated, have been cloned, expressed and purified according to the following procedure.

The cDNA of each proMMP has been cloned in a pET21 vector (Novagen). The resulting vector has been used for the transformation of BL21 cells of Escherichia coli. A culture of the latter transformed cells have been grown in Luria-Bertani medium at a temperature of 37° C. The expression of each protein is induced during the phase of exponential growing by adding of IPTG (isopropyl-beta-D-thiogalactopyranoside) 0.5 mM. Cells have been harvested and lysated, 4 hours later the induction. After the cellular lysis, inclusion bodies have been collected and dissolved in a solution of 6 M urea and 20 mM Tris-HCl at pH 8. Then the proteins have been purified using an Hiprep 16/10 (20 ml) QFF (Pharmacia) with a buffer containing 6 M urea, 20 mM Tris-HCl at pH 8 and eluting with a linear gradient of NaCl until 0.35 M. Every purified protein has been refolded by subsequent dialysis with solutions containing 50 mM Tris-HCl (pH 7.2), 10 mM CaCl₂, 0.1 mM ZnCl₂, 0.3 M NaCl and 0.2 M AHA (Acetohydroxamic acid). During the refolding phases each protein is activated by prodomain cutting. In the final refolding dialysis the AHA concentration is equal to 0.5 M. For the isolation of the single catalytic domain is carried out a chromatographic column Superdex 75 16/60 (Pharmacia) eluted with the buffer of the last dialysis.

The aminoacid sequences of the MMPs, prepared and purified as described above, are reported in the following.

SEQ ID No. 1 MMP-1 P03956
>gi|116852|sp|P03956|MMP1_HUMAN Interstitial
collagenase precursor (Matrix metalloproteinase-1)
(MMP-1) (Fibroblast collagenase)
MHSFPPLLLLLFWGWSHSFPATLETQEQDVDLVQKYLEKYYNLKNDGRQV
EKRRNSGPVVEKLKQMQEFFGLKVTGKPDAETLKVMKQPRCGVPDVAQFV
LTEGNPRWEQTHLTYRIENYTPDLPRADVDHAIEKAFQLWSNVTPLTFTK
VSEGQADIMISFVRGDHRDNSPFDGPGGNLAHAFQPGPGIGGDAHFDEDE
RWTNNFREYNLHRVAAHELGHSLGLSHSTDIGALMYPSYTFSGDVQLAQD
DIDGIQAIYGRSQNPVQPIGPQTPKACDSKLTFDAITTIRGEVMFFKDRF
YMRTNPFYPEVELNFISVFWPQLPNGLEAAYEFADRDEVRFFKGNKYWAV
QGQNVLHGYPKDIYSSFGFPRTVKHIDMLSEENTGKTYFFVANKYWRYDE
YKRSMDPGYPKMIAHDFPGIGHKVDAVFMKDGFFYFFHGTRQYKFDPKTK
RILTLQKANSWFNCRKN
SEQ ID No. 2 MMP-2 P08253
>gi|116856|sp|P08253|MMP2_HUMAN 72 kDa type IV
collagenase precursor (72 kDa gelatinase) (Matrix
metalloproteinase-2) (MMP-2) (Gelatinase A)
(TBE-1)
MEALMARGALTGPLRALCLLGCLLSHAAAAPSPIIKFPGDVAPKTDKELA
VQYLNTFYGCPKESCNLFVLKDTLKKMQKFFGLPQTGDLDQNTIETMRKP
RCGNPDVANYNFFPRKPKWDKNQITYRIIGYTPDLDPETVDDAFARAFQV
WSDVTPLRFSRIHDGEADIMINFGRWEHGDGYPFDGKDGLLAHAFAPGTG
VGGDSHFDDDELWTLGEGQVVRVKYGNADGEYCKFPFLFNGKEYNSCTDT
GRSDGFLWCSTTYNFEKDGKYGFCPHEALFTMGGNAEGQPCKFPFRFQGT
SYDSCTTEGRTDGYRWCGTTEDYDRDKKYGFCPETAMSTVGGNSEGAPCV
FPFTFLGNKYESCTSAGRSDGKMWCATTANYDDDRKWGFCPDQGYSLFLV
AAHEFGHAMGLEHSQDPGALMAPIYTYTKNFRLSQDDIKGIQELYGASPD
IDLGTGPTPTLGPVTPEICKQDIVFDGIAQIRGEIFFFKDRFIWRTVTPR
DKPMGPLLVATFWPELPEKIDAVYEAPQEEKAVFFAGNEYWIYSASTLER
GYPKPLTSLGLPPDVQRVDAAFNWSKNKKTYIFAGDKFWRYNEVKKKMDP
GFPKLIADAWNAIPDNLDAVVDLQGGGHSYFFKGAYYLKLENQSLKSVKF
GSIKSDWLGC
SEQ ID No. 3 MMP-3 P08254
>gi|116857|sp|P08254|MMP3_HUMAN Stromelysin-1
precursor (Matrix metalloproteinase-3) (MMP-3)
(Transin-1) (SL-1)
MKSLPILLLLCVAVCSAYPLDGMRGEDTSMNLVQKYLENYYDLKKDVKQF
VRRKDSGPVVKKIREMQKFLGLEVTGKLDSDTLEVMRKPRCGVPDVGHFR
TFPGIPKWRKTHLTYRIVNYTPDLPKDAVDSAVEKALKVWEEVTPLTFSR
LYEGEADIMISFAVREHGDFYPFDGPGNVLAHAYAPGPGINGDAHFDDDE
QWTKDTTGTNLFLVAAHEIGHSLGLFHSANTEALMYPLYHSLTDLTRFRL
SQDDINGIQSLYGPPPDSPETPLVPTEPVPPEPGTPANCDPALSFDAVST
LRGEILIFKDRHFWRKSLRKLEPELHLISSFWPSLPSGVDAAYEVTSKDL
VFIFKGNQFWAIRGNEVRAGYPRGIHTLGFPPTVRKIDAAISDKEKNKTY
FFVEDKYWRFDEKRNSMEPGFPKQIAEDFPGIDSKIDAVFEEFGFFYFFT
GSSQLEFDPNAKKVTHTLKSNSWLNC
SEQ ID No. 4 MMP-7 P09237
>gi|116861|sp|P09237|MMP7_HUMAN Matrilysin
precursor (Pump-1 protease) (Uterine
metalloproteinase) (Matrix metalloproteinase-7)
(MMP-7) (Matrin)
MRLTVLCAVCLLPGSLALPLPQEAGGMSELQWEQAQDYLKRFYLYDSETK
NANSLEAKLKEMQKFFGLPITGMLNSRVIEIMQKPRCGVPDVAEYSLFPN
SPKWTSKVVTYRIVSYTRDLPHITVDRLVSKALNMWGKEIPLHFRKVVWG
TADIMIGFARGAHGDSYPFDGPGNTLAHAFAPGTGLGGDAHFDEDERWTD
GSSLGINFLYAATHELGHSLGMGHSSDPNAVMYPTYGNGDPQNFKLSQDD
IKGIQKLYGKRSNSRKK
SEQ ID No. 5 MMP-8 P22894
>gi|116862|sp|P22894|MMP8_HUMAN Neutrophil
collagenase precursor (Matrix metalloproteinase-8)
(MMP-8) (PMNL collagenase) (PMNL-CL)
MFSLKTLPFLLLLHVQISKAFPVSSKEKNTKTVQDYLEKFYQLPSNQYQS
TRKNGTNVIVEKLKEMQRFFGLNVTGKPNEETLDMMKKPRCGVPDSGGFM
LTPGNPKWERTNLTYRIRNYTPQLSEAEVERAIKDAFELWSVASPLIFTR
ISQGEADINIAFYQRDHGDNSPFDGPNGILAHAFQPGQGIGGDAHFDAEE
TWTNTSANYNLFLVAAHEFGHSLGLAHSSDPGALMYPNYAFRETSNYSLP
QDDIDGIQAIYGLSSNPIQPTGPSTPKPCDPSLTFDAITTLRGEILFFKD
RYFWRRHPQLQRVEMNFISLFWPSLPTGIQAAYEDFDRDLIFLFKGNQYW
ALSGYDILQGYPKDISNYGFPSSVQAIDAAVFYRSKTYFFVNDQFWRYDN
QRQFMEPGYPKSISGAFPGIESKVDAVFQQEHFFHVFSGPRYYAFDLIAQ
RVTRVARGNKWLNCRYG
SEQ ID No. 6 MMP-9 P14780
>gi|116863|sp|P14780|MMP9_HUMAN Matrix metallo-
proteinase-9 precursor (MMP-9) (92 kDa type
IV collagenase) (92 kDa gelatinase) (Gelatinase B)
(GELB) [Contains: 67 kDa matrix metalloproteinase-
9; 82 kDa matrix metalloproteinase-9]
MSLWQPLVLVLLVLGCCFAAPRQRQSTLVLFPGDLRTNLTDRQLAEEYLY
RYGYTRVAEMRGESKSLGPALLLLQKQLSLPETGELDSATLKAMRTPRCG
VPDLGRFQTFEGDLKWHHHNITYWIQNYSEDLPRAVIDDAFARAFALWSA
VTPLTFTRVYSRDADIVIQFGVAEHGDGYPFDGKDGLLAHAFPPGPGIQG
DAHFDDDELWSLGKGVVVPTRFGNADGAACHFPFIFEGRSYSACTTDGRS
DGLPWCSTTANYDTDDRFGFCPSERLYTRDGNADGKPCQFPFIFQGQSYS
ACTTDGRSDGYRWCATTANYDRDKLFGFCPTRADSTVMGGNSAGELCVFP
FTFLGKEYSTCTSEGRGDGRLWCATTSNFDSDKKWGFCPDQGYSLFLVAA
HEFGHALGLDHSSVPEALMYPMYRFTEGPPLHKDDVNGIRHLYGPRPEPE
PRPPTTTTPQPTAPPTVCPTGPPTVHPSERPTAGPTGPPSAGPTGPPTAG
PSTATTVPLSPVDDACNVNIFDAIAEIGNQLYLFKDGKYWRFSEGRGSRP
QGPFLIADKWPALPRKLDSVFEEPLSKKLFFFSGRQVWVYTGASVLGPRR
LDKLGLGADVAQVTGALRSGRGKMLLFSGRRLWRFDVKAQMVDPRSASEV
DRMFPGVPLDTHDVFQYREKAYFGQDRFYWRVVSSRSELNQVDQVGYVTY
DILQCPED
SEQ ID No. 7 MMP-10 P09238
>gi|116869|sp|P09238|MMP10_HUMAN Stromelysin-2
precursor (Matrix metalloproteinase-10) (MMP-10)
(Transin-2) (SL-2)
MMHLAFLVLLCLPVCSAYPLSGAAKEEDSNKDLAQQYLEKYYNLEKDVKQ
FRRKDSNLIVKKIQGMQKFLGLEVTGKLDTDTLEVMRKPRCGVPDVGHFS
SFPGMPKWRKTHLTYRIVNYTPDLPRDAVDSAIEKALKVWEEVTPLTFSR
LYEGEADIMISFAVKEHGDFYSFDGPGHSLAHAYPPGPGLYGDIHFDDDE
KWTEDASGTNLFLVAAHELGHSLGLFHSANTEALMYPLYNSFTELAQFRL
SQDDVNGIQSLYGPPPASTEEPLVPTKSVPSGSEMPAKCDPALSFDAIST
LRGEYLFFKDRYFWRRSHWNPEPEFHLISAFWPSLPSYLDMYEVNSRDTV
FIFKGNEFWAIRGNEVQAGYPRGIHTLGFPPTIRKIDAAVSDKEKKKTYF
FMDKYWRFDENSQSMEQGFPRLIADDFPGVEPKVDAVLQAFGFFYFFSGS
SQFEFDPNARMVTHILKSNSWLHC
SEQ ID No. 8 MMP-11 P24347
>gi|116871|sp|P24347|MMP11 _HUMAN Stromelysin-3
precursor (Matrix metalloproteinase-11) (MMP-11)
(ST3) (SL-3)
MAPMWLRSAAARALLPPMLLLLLQPPPLLARALPPDVHHLHAERRGPQPW
HAALPSSPAPAPATQEAPRPASSLRPPRCGVPDPSDGLSARNRQKRFVLS
GGRWEKTDLTYRILRFPWQLVQEQVRQTMAEALKVWSDVTPLTFTEVHEG
RADIMIDFARYWDGDDLPFDGPGGILAHAFFPKTHREGDVHFDYDETWTI
GDDQGTDLLQVAAHEFGHVLGLQHTTAAKALMSAFYTFRYPLSLSPDDCR
GVQHLYGQPWPTVTSRTPALGPQAGIDTNEIAPLEPDAPPDACEASFDAV
STIRGELFFFKAGFVWRLRGGQLQPGYPALASRHWQGLPSPVDAAFEDAQ
GHIWFFQGAQYWVYDGEKPVLGPAPLTELGLVRFPVHAALVWGPEKNKIY
FFRGRDYVVRFHPSTRRVDSPVPRRATDWRGVPSEIDAAFQDADGYAYFL
RGRLYWKFDPVKVKALEGFPRLVGPDFFGCAEPANTFL
SEQ ID No. 9 MMP-12 P39900
>gi|729179|sp|P39900|MMP12_HUMAN Macrophage
metalloelastase precursor (HME) (Matrix
metalloproteinase-12) (MMP-12) (Macrophage
elastase) (ME)
MKFLLILLLQATASGALPLNSSTSLEKNNVLFGERYLEKFYGLEINKLPV
TKMKYSGNLMKEKIQEMQHFLGLKVTGQLDTSTLEMMHAPRCGVPDVHHF
REMPGGPVWRKHYITYRINNYTPDMNREDVDYAIRKAFQVWSNVTPLKFS
KINTGMADILVVFARGAHGDFHAFDGKGGILAHAFGPGSGIGGDAHFDED
EFWTTHSGGTNLFLTAVHEIGHSLGLGHSSDPKAVMFPTYKYVDINTFRL
SADDIRGIQSLYGDPKENQRLPNPDNSEPALCDPNLSFDAVTTVGNKIFF
FKDRFFWLKVSERPKTSVNLISSLWPTLPSGIEAAYEIEARNQVFLFKDD
KYWLISNLRPEPNYPKSIHSFGFPNFVKKIDAAVFNPRFYRTYFFVDNQY
WRYDERRQMMDPGYPKLITKNFQGIGPKIDAVFYSKNKYYYFFQGSNQFE
YDFLLQRITKTLKSNSWFGC
SEQ ID No. 10 MMP-13 P45452
>gi|1168998|sp|P45452|MMP13_HUMAN Collagenase 3
precursor (Matrix metalloproteinase-13) (MMP-13)
MHPGVLAAFLFLSWTHCRALPLPSGGDEDDLSEEDLQFAERYLRSYYHPT
NLAGILKENAASSMTERLREMQSFFGLEVTGKLDDNTLDVMKKPRCGVPD
VGEYNVFPRTLKWSKMNLTYRIVNYTPDMTHSEVEKAFKKAFKVWSDVTP
LNFTRLHDGIADIMISFGIKEHGDFYPFDGPSGLLAHAFPPGPNYGGDAH
FDDDETWTSSSKGYNLFLVAAHEFGHSLGLDHSKDPGALMFPIYTYTGKS
HFMLPDDDVQGIQSLYGPGDEDPNPKHPKTPDKCDPSLSLDAITSLRGET
MIFKDRFFWRLHPQQVDAELFLTKSFWPELPNRIDAAYEHPSHDLIFIFR
GRKFWALNGYDILEGYPKKISELGLPKEVKKISAAVHFEDTGKTLLFSGN
QVWRYDDTNHIMDKDYPRLIEEDFPGIGDKVDAVYEKNGYIYFFNGPIQF
EYSIWSNRIVRVMPANSILWC
SEQ ID No. 11 MMP-14 P50281
>gi|60392771|sp|P50281|MMP14_HUMAN Matrix metallo-
proteinase-14 precursor (MMP-14) (Membrane-type
matrix metalloproteinase 1) (MT-MMP 1) (MTMMP1)
(Membrane-type-i matrix metalloproteinase)
(MT1-MMP) (MT1MMP) (MMP-X1)
MSPAPRPSRCLLLPLLTLGTALASLGSAQSSSFSPEAWLQQYGYLPPGDL
RTHTQRSPQSLSAAIAAMQKFYGLQVTGKADADTMKAMRRPRCGVPDKFG
AEIKANVRRKRYAIQGLKWQHNEITFCIQNYTPKVGEYATYEAIRKAFRV
WESATPLRFREVPYAYIREGHEKQADIMIFFAEGFHGDSTPFDGEGGFLA
HAYFPGPNIGGDTHFDSAEPWTVRNEDLNGNDIFLVAVHELGHALGLEHS
SDPSAIMAPFYQWMDTENFVLPDDDRRGIQQLYGGESGFPTKMPPQPRTT
SRPSVPDKPKNPTYGPNICDGNFDTVAMLRGEMFVFKERWFWRVRNNQVM
DGYPMPIGQFWRGLPASINTAYERKDGKFVFFKGDKHWVFDEASLEPGYP
KHIKELGRGLPTDKIDAALFWMPNGKTYFFRGNKYYRFNEELRAVDSEYP
KNIKVWEGIPESPRGSFMGSDEVFTYFYKGNKYWKFNNQKLKVEPGYPKS
ALRDWMGCPSGGRPDEGTEEETEVIIIEVDEEGGGAVSAAAVVLPVLLLL
LVLAVGLAVFFFRRHGTPRRLLYCQRSLLDKV
SEQ ID No. 12 MMP-15 P51511
>gi|705988|sp|P51511|MMP15_HUMAN Matrix metallo-
proteinase-15 precursor (MMP-1 ) (Membrane-type
matrix metalloproteinase 2) (MT-MMP 2) (MTMMP2)
(Membrane-type-2 matrix metalloproteinase)
(MT2-MMP) (MT2MMP) (SMCP-2)
MGSDPSAPGRPGWTGSLLGDREEMRPRLLPLLLVLLGCLGLGVAAEDAEV
HAENWLRLYGYLPQPSRHMSTMRSAQILASALAEMQRFYGIPVTGVLDEE
TKEWMKRPRCGVPDQFGVRVKANLRRRRKRYALTGRKWNNHHLTFSIQNY
TEKLGWYHSMEAVRRAFRVWEQATPLVFQEVPYEDIRLRRQKEADIMVLF
ASGFHGDSSPFDGTGGFLAHAYFPGPGLGGDTHFDADEPWTFSSTDLHGN
NLFLVAVHELGHALGLEHSSNPNAIMAPFYQWKDVDNFKLPEDDLRGIQQ
LYGTPDGQPQPTQPLPTVTPRRPGRPDHRPPRPPQPPPPGGKPERPPKPG
PPVQPRATERPDOYGPNICDGDFDTVAMLRGEMFVFKGRWFWRVRHNRVL
DNYPMPIGHFWRGLPGDISAAYERQDGRFVFFKGDRYWLFREANLEPGYP
QPLTSYGLGIPYDRIDTAIWWEPTGHTFFFQEDRYWRFNEETQRGDPGYP
KPISVWQGIPASPKGAFLSNDAAYTYFYKGTKYWKFDNERLRMEPGYPKS
ILRDFMGCQEHVEPGPRWPDVARPPFNPHGGAEPGADSAEGDVGDGDGDF
GAGVNKDGGSRVVVQMEEVARTVNVVMVLVPLLLLLCVLGLTYALVQMQR
KGAPRVLLYCKRSLQEWV
SEQ ID No. 13 MMP-16 P51512
Isoform 1
>gi|3041669|sp|P51512|MMP16_HUMAN Matrix metallo-
proteinase-16 precursor (MMP-16) (Membrane-type
matrix metalloproteinase 3) (MT-MMP 3) (MTMMP3)
(Membrane-type-3 matrix metalloproteinase)
(MT3-MMP) (MT3MMP) (MMP-X2)
MILLTFSTGRRLDFVHHSGVFFLQTLLWILCATVCGTEQYFNVEVWLQKY
GYLPPTDPRMSVLRSAETMQSALAAMQQFYGINMTGKVDRNTIDWMKKPR
CGVPDOTRGSSKFHIRRKRYALTGQKWQHKHITYSIKNVTPKVGDPETRK
AIRRAFDVWQNVTPLTFEEVPYSELENGKRDVDITIIFASGFHGDSSPFD
GEGGFLAHAYFPGPGIGGDTHFDSDEPWTLGNPNHDGNDLFLVAVHELGH
ALGLEHSNDPTAIMAPFYQYMETDNFKLPNDDLQGIQKIYGPPDKIPPPT
RPLPTVPPHRSIPPADPRKNDRPKPPRPPTGRPSYPGAKPNICDGNFNTL
AILRREMFVFKDQWFWRVRNNRVMDGYPMQITYFWRGLPPSIDAVYENSD
GNFVFFKGNKYWVFKDTTLQPGYPHDLITLGSGIPPHGIDSAIWWEDVGK
TYFFKGDRYWRYSEEMKTMDPGYPKPITVWKGIPESPQGAFVHKENGFTY
FYKGKEYWKFNNQILKVEPGYPRSILKDFMGCDGPTDRVKEGHSPPDDVD
IVIKLDNTASTVKAIAIVIPCILALCLLVLVYTVFQFKRKGTPRHILYCK
RSMQEWV
SEQ ID No. 14
Isoform 2
>gi|13027800|ref|NP_072086.1| matrix metallo-
proteinase 16 isoform 2 [Homo sapiens]
MILLTFSTGRRLDFVHHSGVFFLQTLLWILCATVCGTEQYFNVEVWLQKY
GYLPPTDPRMSVLRSAETMQSALAAMQQFYGINMTGKVDRNTIDWMKKPR
CGVPDQTRGSSKFHIRRKRYALTGQKWQHKHITYSIKNVTPKVGDPETRK
AIRRAFDVWQNVTPLTFEEVPYSELENGKRDVDITIIFASGFHGDSSPFD
GEGGFLAHAYFPGPGIGGDTHFDSDEPWTLGNPNHDGNDLFLVAVHELGH
ALGLEHSNDPTAIMAPFYQYMETDNFKLPNDDLQGIQKIYGPPDKIPPPT
RPLPTVPPHRSIPPADPRKNDRPKPPRPPTGRPSYPGAKPNICDGNFNTL
AILRREMFVFKDQWFWRVRNNRVMDGYPMQITYFWRGLPPSIDAVYENSD
GNFVFFKVKGDTLSVIQDGWLYKYHWKWILEQRQSVPVLSRQTEKHKTYE
ELSSITY
SEQ ID No. 15 MMP-17 Q9ULZ9
>gi|21264469|sp|Q9ULZ9|MMP17_HUMAN Matrix metallo-
proteinase-17 precursor (MMP-17) (Membrane-type
matrix metalloproteinase 4) (MT-MMP 4) (Membrane-
type-4 matrix metalloproteinase) (MT4-MMP)
MRRRAARGPGPPPPGPGLSRLPLLPLPLLLLLALGTRGGCAAPAPAPRAE
DLSLGVEWLSRFGYLPPADPTTGQLQTQEELSKAITAMQQFGGLEATGIL
DEATLALMKTPRCSLPDLPVLTQARRRRQAPAPTKWNKRNLSWRVRTFPR
DSPLGHDTVRALMYYALKVWSDIAPLNFHEVAGSTADIQIDFSKADHNDG
YPFDGPGGTVAHAFFPGHHHTAGDTHFDDDEAWTFRSSDAHGMDLFAVAV
HEFGHAIGLSHVAAAHSIMRPYYQGPVGDPLRYGLPYEDKVRVWQLYGVR
ESVSPTAQPEEPPLLPEPPDNRSSAPPRKDVPHRCSTHFDAVAQIRGEAF
FFKGKYFWRLTRDRHLVSLQPAQMHRFWRGLPLHLDSVDAVYERTSDHKI
VFFKGDRYWVFKDNNVEEGYPRPVSDFSLPPGGIDMFSWAHNDRTYFFKD
QLYWRYDDHTRHMDPGYPAQSPLWRGVPSTLDDAMRWSDGASYFFRGQEY
WKVLDGELEVAPGYPOSTARDWLVCGDSQADGSVMGVDMEGPRAPPGQHD
QSRSEDGYEVCSCTSGASSPPGAPGPLVAATMLLLLPPLSPGALWTAAQA
LTL
SEQ ID No. 16 MMP-19 Q99542
Isoform rasi-1
>gi|12643345|sp|Q99542|MMP19_HUMAN Matrix metallo-
proteinase-19 precursor (MMP-19) (Matrix metallo-
proteinase RASI) (MMP-18)
MNCQOLWLGFLLPMTVSGRVLGLAEVAPVDYLSQYGYLQKPLEGSNNFKP
EDITEALRAFQEASELPVSGQLDDATRARMRQPRCGLEDPFNQKTLKYLL
LGRWRKKHLTFRILNLPSTLPPHTARAALRQAFQDWSNVAPLTFQEVQAG
MDIRLSFHGRQSSYCSNTFDGPGRVLAHADIPELGSVHFDEDEFWTEGTY
RGVNLRIIAAHEVGHALGLGHSRYSQALMAPVYEGYRPHFKLHPDDVAGI
QALYGKKSPVIRDEEEEETELPTVPPVPTEPSPMPDPCSSELDAMMLGPR
GKTYAFKGDYVWTVSDSGPGPLFRVSALWEGLPGNLDMVYSPRTQWIHFF
KGDKVVVRYINFKMSPGFPKKLNRVEPNLDAALYWPLNQKVFLFKGSGYW
QWDELARTDFSSYPKPIKGLFTGVPNQPSAAMSWQDGRVYFFKGKVYWRL
NQQLRVEKGYPRNISHNWMHCRPRTIDTTPSGGNTTPSGTGITLDTTLSA
TETTFEY
SEQ ID No. 17
Isoforma rasi-6
>gi|13027792|ref|NP_073628.1|matrix metallo-
proteinase 19 isoform rasi-6 [Homo sapiens]
MRQPRCGLEDPFNQKTLKYLLLGRWRKKHLTFRILNLPSTLPPHTARAAL
RQAFQDWSNVAPLTFQEVQAGAADIRLSFHGRQSSYCSNTFDGPGRVLAH
ADIPELGSVHFDEDEFWTEGTYRGVNLRIIAAHEVGHALGLGHSRYSQAL
MAPVYEGYRPHFKLHPDDVAGIQALYGKKSPVIRDEEEEETELPTVPPVP
TEPSPMPDPCSSELDAMMLGEAPPLQAVGRRWGQPADPEAWTNGSDMGLQ
HEQWRAPWEDLCFQGGLCVDCIRFRTGPLVPSVCPLGGAPRKPGCCCLLA
SNTMDSLL
SEQ ID No. 18 MMP-20 O60882
>gi|56405303|sp|O60882|MMP20_HUMAN Matrix metallo-
proteinase-20 precursor (MMP-20) (Enamel metallo-
proteinase) (Enamelysin)
MKVLPASGLAVFLIMALTFSTAAPSLVAASPRTWRNNYRLAQAYLDKYYT
NKEGHQIGEMVARGSNSMIRKIKELQAFFGLQVTGKLDQTTMNVIKKPRC
GVPDVANYRLFPGEPKWKKNTLTYRISKYTPSMSSVEVDKAVEMALQAWS
SAVPLSFVRINSGEADIMISFENGDHGDSYPFDGPRGTLAHAFAPGEGLG
GDTHFDNAEKWTMGTNGFNLFTVAAHEFGHALGLAHSTDPSALMYPTYKY
KNPYGFHLPKDDVKGIQALYGPRKVFLGKPTLPHAPHHKPSIPDLCDSSS
SFDAVTMLGKELLLFKDRIFWRRQVHLRTGIRPSTITSSFPQLMSNVDAA
YEVAERGTAYFFKGPHYWITRGFQMQGPPRTIYDFGFPRHVQQIDAAVYL
REPQKTLFFVGDEYYSYDERKRKMEKDYPKNTEEEFSGVNGQIDAAVELN
GYIYFFSGPKTYKYDTEKEDVVSVVKSSSWIGC
SEQ ID No. 19 MMP-21 Q8N119
>gi|50401068|sp|Q8N119|MMP21_HUMAN Matrix metallo-
proteinase-21 precursor (MMP-21)
MLAASIFRPTLLLCWLAAPWPTQPESLFHSRDRSDLEPSPLRQAKPIADL
HAAQRFLSRYGWSGVWAAWGPSPEGPPETPKGAALAEAVRRFQRANALPA
SGELDAATLAAMNRPRCGVPDMRPPPPSAPPSPPGPPPRARSRRSPRAPL
SLSRRGWQPRGYPDGGMQAFSKRTLSWRLLGEALSSQLSAADQRRIVALA
FRMWSEVTPLDFREDLAAPGAAVDIKLGFGRGRHLGCPRAFDGSGQEFAH
AWRLGDIHFDDDEHFTPPTSDTGISLLKVAVHEIGHVLGLPHTYRTGSIM
QPNYIPQEPAFELDWSDRKAIQKLYGSCEGSFDTAFDWIRKERNQYGEVM
VRFSTYFFRNSWYWLYENRNNRTRYGDPIQILTGWPGIPTHNIDAFVHIW
TWKRDERYFFQGNQYWRYDSDKDQALTEDEQGKSYPKLISEGFPGIPSPL
DTAFYDRRQKLIYFFKESLVFAFDVNRNRVLNSYPKRITEVFPAVIPQNH
PFRNIDSAYYSYAYNSIFFFKGNAYWKVVNDKDKQONSWLPANGLFPKKF
ISEKWFDVCDVHISTLNM
SEQ ID No. 20 MMP-23A O75900
>gi|4758730|ref|NP_004650.11 matrix metallo-
proteinase 23A [Homo sapiens]
MGRGARVPSEAPGAGVERRWLGAALVALCLLPALVLLARLGAPAVPAWSA
AQGDVAALGLSAVPPTRVPGPLAPRRRRYTLTPARLRWDHLNLTYRILSF
PRNLLSPRETRRALAAAFRMWSDVSPFSFREVAPEQPSDLRIGFYPINHT
DCLVSALHHCFDGPTGELAHAFFPPHGGIHFDDSEYWVLGPTRYSWKKGV
WLTDLVHVAAHEIGHALGLMHSQHGRALMHLNATLRGWKALSQDELWGLH
RLYGCLDRLFVCASWARRGFCDARRRLMKRLCPSSCDFCYEFPFPTVATT
PPPPRTKTRLVPEGRNVTFRCGQKILHKKGKVYWYKDQEPLEFSYPGYLA
LGEAHLSIIANAVNEGTYTCVVRRQQRVLTTYSWRVRVRG
SEQ ID No. 21 MMP-23B Q9UBR9
>gi|4468604|emb|CAB38176.1|MMP-23 [Homo sapiens]
MGRGARVPSEAPGAGVERRWLGMLVALCLLPALVLLARLGAPAVPAWSMQ
GDVAALGLSAVPPTRVPGPLAPRRRRYTLTPARLRWDHFNLTYRILSFPR
NLLSPRETRRALAAAFRMWSDVSPFSFREVAPEQPSDLRIGFYPINHTDC
LVSALHHCFDGPTGELAHAFFPPHGGIHFDDSEYWVLGPTRYSWKKGVWL
TDLVHVAAHEIGHALGMHSQHGRALMHLNALRGWKALSQDELWGLHRLYG
CLDRLFVCASWARRGFCDARRRLMKRLCPSSCDFCYEFPFPTVATTPPPP
RTKTRLVPEGRNVTFRCGQKILHKKGKVYWYKDQEPLEFSYPGYLALGEA
HLSIIANAVNEGTYTCVVRRQQRVLTTYSWRVRVRG
SEQ ID No. 22 MMP-24 Q9Y5R2
>gi|12585280|sp|Q9Y5R2|MMP24_HUMAN Matrix metallo-
proteinase-24 precursor (MMP-24) (Membrane-type
matrix metalloproteinase 5) (MT-MMP 5) (Membrane-
type-5 matrix metalloproteinase) (MT5-MMP)
MPRSRGGRAAPGPPPPPPPPGQAPRWSRWRVPGRLLLLLLPALCCLPGAA
RAAAAAAGAGNRAAVAVAVARADEAEAPFAGQNWLKSYGYLLPYDSRASA
LHSAKALQSAVSTMQQFYGIPVTGVLDQTTIEWMKKPRCGVPDHPHLSRR
RRNKRYALTGQKWRQKHITYSIHNYTPKVGELDTRKIRQAFDVWQKVPLT
FEEVPYHEIKSDRKEADIMIFFASGFHGDSSPFDGEGGFLAHAYFPGPGI
GGDTHFDSDEPWTLGNANHDGNDLFLVAVHELGHALGLEHSSDPSAIMAP
FYQYMETHNFKLPODDLQGIQKIYGPPAEPLEPTRPLPTLPVRRIHSPSE
RKHERQPRPPRPPLGDRPSTPGTKPNICDGNFNTVALFRGEMFVFKDRWF
WRLRNNRVQEGYPMQIEQFWKGLPARIDAAYERADGRFVFFKGDKYWVFK
EVTVEPGYPHSLGELGSCLPREGIDTALRWEPVGKTYFFKGERYWRYSEE
RRATDPGYPKPITVWKGIPQAPQGAFISKEGYYTYFYKGRDYWKFDNQKL
SVEPGYPRNILRDWMGCNQKEVERRKERRLPQDDVDIMVTINDVPGSVNA
VAVVIPCILSLCILVLVYTIFQFKNKTGPQPVTYYKRPVQEWV
SEQ ID No. 23 MMP-25 Q9NPA2
>gi|2585274|sp|Q9NPA2|MMP25_HUMAN Matrix metallo-
proteinase-25 precursor (MMP-25) (Membrane-type
matrix metalloproteinase 6) (MT-MMP 6) (Membrane-
type-6 matrix metalloproteinase) (MT6-MMP)
(Leukolysin)
MRLRLRLLALLLLLLAPPARAPKPSAQDVSLGVDWLTRYGYLPPPHPAQA
QLQSPEKLRDAIKVMQRFAGLPETGRMDPGTVATMRKPRCSLPDVLGVAG
LVRRRRRYALSGSVWKKRTLTWRVRSFPQSSQLSQEVRVLMSYALMAWGM
ESGLTFHEVDSPQGQEPDILIDFARAFHQDSYPFDGLGGTLAHAFFPGEH
PISGDTHFDDEETTFGSKDGEGTDLFAVAVHEFGHALGLGHSSAPNSIMR
PFYQGPVGDPDKYRLSQDDRDGLQQLYGKAPQTPYDKPTRKPLAPPPQPP
ASPTHSPSFPIPDRCEGNFDAIANIRGETFFFKGPWFWRLQPSGQLVSPR
PARLHRFWEGLPAQVRWQMYARHRDGRILLFSGPQFWVFQDRQLEGGARP
LTELGLPPGEEVDAVFSWPQNGKYLVRGROYWRYDEAAARPDPGYPRDLS
LWEGAPPSPDDVTVSNAGDWFYWRFPKNSIKTEPDAPQPMGPNWLDCPAP
SSGPRAPRPPKATPVSETCDCQCELNQMGRWPAPIPLLLLPLLVGGVASR
SEQ ID No. 24 MMP-26 Q9NRE1
>gi|136294931sp|Q9NRE1|MMP26_HUMAN Matrix metallo-
proteinase-26 precursor (MMP-26) (Matrilysin-2)
(Endometase)
MQLVILRVTIFLPWCFAVPVPPAADHKGWDFVEGYFHQFFLTKKESPLLT
QETQTQLLQQFHRNGTDLLDMQMHALLHQPHCGVPDGSDTSISPGRCKWN
KHTLTYRIINYPHDMKPSAVKDSIYNAVSIWSNVTPLIFQQVQNGDADIK
VSFWQWAHEDGWPFDGPGGILGHAFLPNSGNPGWHFDKNEHWSASDTGYN
LFLVATHEIGHSLGLQHSGNQSSIMYPTYWYHDPRTFQLSADDIQRIQHL
YGEKCSSDIP
SEQ ID No. 25 MMP-27 Q9H306
>gi|11066090|gb|AAG28453.1|AF195192_1 matrix
metalloprotease MMP-27 [Homo sapiens]
MKRLLLLFLFFITFSSAFPLVRMMENEENVQLAQAYLNQFYSLEIEGNHL
VQSKNRSLIDDKIREMQAFFGLTVTGKLDSNTLEIMKTPRCGVPDVGQYG
YTLPGWRKYNLTYRIINYTPDMARAAVDEAIQEGLEVWSKVTPLKFTKIS
KGIADIMIAFRTRVHGRCPRYFDGPLGVLGHAFPPGPGLGGDTHFDEDEN
WTKDGAGFNLFLVAAHEFGHALGLSHSNDQTALMFPNYVSLDPRKYPLSQ
DDINGIQSIYGGLPKEPAKPKEPTIPHACDPDLTFDAITTFRREVMFFKG
RHLWRIYYDITDVEFELIASFWPSLPADLQAAYENPRDKILVFKDENFWM
IRGYAVLPDYPKSIHTLGFPGRVKKIDAAVCDKTTRKTYFFVGIWCWRFD
EMTQTMDKGFPQRWKHFPGSIRVDAAFQYKGFFFFSRGSKQFEYDIKTKN
ITRIMRTNTWFQCKEPKNSSFGFDINKEKAHSGGIKILYHKSLSLFIFGI
VHLLKNTSIYQ
SEQ ID No. 26 MMP-28 Q9H239
Isoform 1
>gi|37538314|sp|Q9H239|MMP28_HUMAN Matrix metallo-
proteinase-28 precursor (MMP-28) (Epilysin)
(UNQ1893/PR04339)
MVARVGLLLRALQLLLWGHLDAQPAERGGQELRKEAEAFLEKYGYLNEQV
PKAPTSTRFSDAIRAFQWVSQLPVSGVLDRATLRQMTRPRCGVTDTNSYM
WAERISDLFARHRTKMRRKKRFAKQGNKWYKQHLSYRLVNWPEHLPEPAV
RGAVRAAFQLWSNVSALEFWEAPATGPADIRLTFFQGDHNDGLGNAFDGP
GGALAHAFLPRRGEAHFDQDERWSLSRRRGRNLFVVLAHEIGHTLGLTHS
PAPRALMAPYYKRLGRDALLSWDDVLAVQSLYGKPLGGSVAVQLPGKLFT
DFETWDSYSPQGRRPETQGPKYCHSSFDAITVDRQQQLYIFKGSHFWEVA
ADGNVSEPRPLQERWVGLPPNIEAAAVSLNDGDFYFFKGGRCWRFRGPKP
VWGLPQLCRAGGLPRHPDAALFFPPLRRLILFKGARYYVLARGGLQVEPY
YPRSLQDWGGIPEEVSGALPRPDGSIIFFRDDRYWRLDQAKLQATTSGRW
ATELPWMGCWHANSGSALF
SEQ ID No. 27
Isoform 2
>gi|14589912|ref|NP_116568.1|matrix metallo-
proteinase 28 preproprotein isoform 2 [Homo
sapiens]
MVARVGLLLRALQLLLWGHLDAQPAERGGQELRKEAEAFLEKYGYLNEQV
PKAPTSTRFSDAIRAFQWVSQLPVSGVLDRATLRQMTRPRCGVTDTNSYA
AWAERISDLFARHRTKMRRKKRFAKQGNKWYKQHLSYRLVNWPEHLPEPA
VRGAVRMFQLWSNVSALEFWEAPATGPADIRLTFFQGDHNDGLGNAFDGP
GGLALAHAFLPRRGEAHFDQDERWSLSRRRGRNLFVVLAHEIGHTHGLTH
SPAPRALMAPYYKRLGRDALLSWDDVLAVQSLYGKPLGGSVAVQLPGKLF
TDFETWDSYSPOGRRPETQGPKYCHSSFDAITVDRQQQLYIFKGSHFWEV
AADGNVSEPRPLQERWVGLPPNIEMAVSLNDGDFYFFKVQSV
SEQ ID No. 28 MMP-like1 O43923
>gi|4758728|ref|NP_004133.1| matrix metallo-
proteinase-like 1 [Homo sapiens]
MDPGTVATMRKPRCSLPDVLGVAGLVRRRRRYALSGSVWKKRTLTWRVRS
FPQSSQLSQETVRVLMSYALMAWGMESGLTFHEVDSPQGQEPDILIDFAR
AFHQDSYPFDGLGGTLAHAFFPGEHPISGDTHFDDEETWTFGSKASQQLE
QELAGGSPVDEELGFSRGWRVNPLGPGSPERLS

### Example 3

### Evaluation of the Mutated MMP-3 Stability (SEQ ID No. 3)

Studies have been carried out to evaluate stability of the wild type human metalloproteinase 3 (MMP-3) (SEQ ID No. 3) catalytic domain and of the mutated human metalloproteinase 3 (MMP-3) (SEQ ID No. 3) catalytic domain, wherein Phenylalanine has been mutated in Aspartate. The analysis has been carried out comparing stability towards autoproteolysis of the two proteins in the same conditions.

The two proteins have been prepared following the procedure reported above in Example 1, and the stability has been analysed by SDS/polyacrylamide gel electrophoresis according to the following procedure.

A sample of the protein is used, dissolved in a buffer containing Tris hydroxymethylaminoethane (Tris) 50 mM pH=7.2; CaCl₂ 5 mM; ZnCl₂ 0.1 mM; NaCl 0.3 M; and acetohydroxamic acid (AHA) 0.5 M, and having a concentration of 0.45 mM. The sample is maintained at room temperature.

At intervals corresponding to 0, 4, 7, 11 and 14 days 5 μL aliquots of the two samples maintained at room temperature are collected and immediately frozen at −80°; all the collected samples having the above said protein concentration are analysed by electrophoresis on SDS/polyacrylamide gel (17%), thus obtaining the results showed in FIG. 1 and illustrated in the following.

The analysis shows a higher stability of the mutated protein in respect of the wild-type protein. In fact, both wild-type and mutated proteins show a degradation band since time=0, but during the next 14 days the intensity of the band corresponding to the non degraded wild-type protein shows a significant decrease. On the contrary, the intensity of the band corresponding to the non degraded mutated protein is substantially constant during 14 days. Moreover, the fragmentation of wild-type protein involving the aminoacidic residue 171 corresponding to the mutation site in the mutated protein yields to a fragment with lower molecular weight if compared with the fragment obtained for the mutated protein. This indicates different cutting sites for wild-type and mutated proteins, so that the mutation site of the mutated protein is less preferred as cutting site than that in the same position of the wild-type protein, this being a suggestion that the non degraded mutated protein is more stable.

Finally, as showed in FIG. 1, the band corresponding to the fragment coming from degradation of the mutated protein has an intensity which is substantially maintained, and this indicates that the mutated protein is not only less susceptible to fragmentation but the fragment coming from its fragmentation is also more stable than that forming from fragmentation of the wild-type protein.

### Example 4

### Evaluation of Stability of the Mutated MMP-10 (SEQ ID No. 7)

Studies have been carried out on stability of the wild type human metalloproteinase 10 (MMP-10) (SEQ ID No. 7) catalytic domain and of the mutated human metalloproteinase 10 (MMP-10) (SEQ ID No. 7) catalytic domain, wherein Phenylalanine has been mutated in Aspartate. The analysis has been carried out comparing stability towards autoproteolysis of the two proteins in the same conditions.

The two proteins have been prepared following the procedure reported above in Example 1, and the stability has been analysed by SDS/polyacrylamide gel electrophoresis according to the following procedure.

A sample of the protein is used, dissolved in a buffer containing Tris 10 mM pH=7.2; CaCl₂ 10 mM; ZnCl₂ 0.1 mM; NaCl 0.3 M; and AHA 0.5 M, and having a concentration of 0.3 mM for the wild-type protein and of 0.26 mM for the mutated protein. The samples are maintained at 4° C.

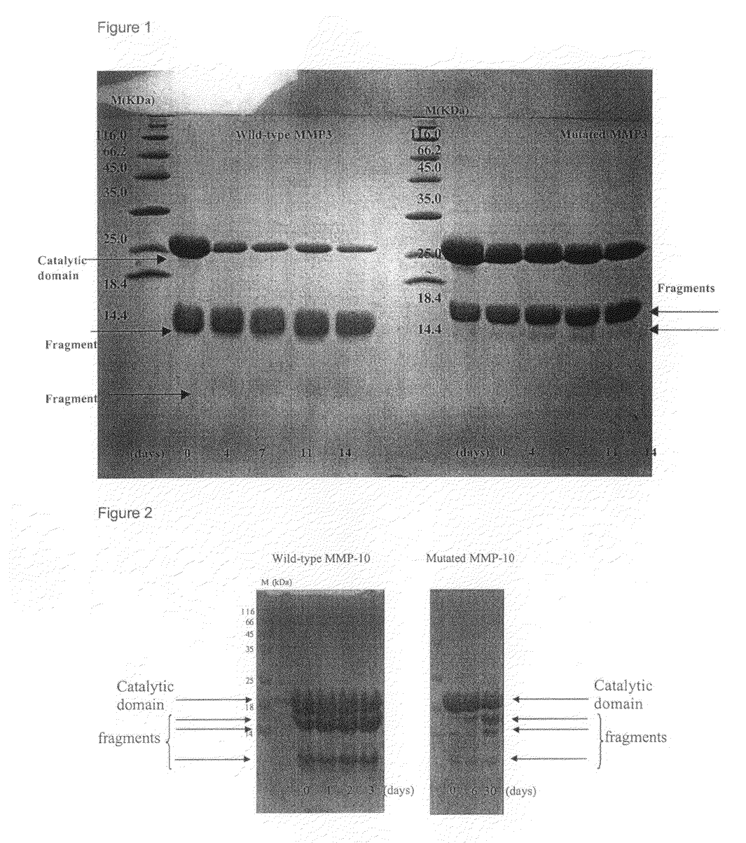

At intervals corresponding to 0, 1, 2, and 3 days for the wild-type protein and 0, 6 and 30 days for the mutated protein, 5 μL aliquots of the two samples maintained at 4° C. are collected and immediately frozen at −80° C.; all the collected samples having the above said protein concentrations, are analysed by electrophoresis SDS/polyacrylamide gel (17%), thus obtaining the results showed in FIG. 2. Besides the observations already made above for the results concerning MMP-3 (SEQ ID No. 3), that are substantially the same results as those obtained for MMP-10 (SEQ ID No. 10), it is significant to notice the high stability showed by the mutated protein even after 30 days.

### Example 5

### Evaluation of Stability of the Mutated MMP-13 (SEQ ID No. 10)

Studies have been carried out on the stability of the wild type human metalloproteinase 13 (MMP-13) (SEQ ID No. 10) catalytic domain and of the mutated human metalloproteinase 13 (MMP-13) (SEQ ID No. 10) catalytic domain where phenylalanine has been mutated in aspartate. The analysis has been carried out comparing stability towards autoproteolysis of the two proteins in the same conditions.

The two proteins have been prepared following the procedure reported above in Example 1, and the stability has been analysed by SDS/polyacrylamide gel electrophoresis according to the following procedure.

A sample of the protein is used, dissolved in a buffer containing Tris 50 mM pH=7.2; CaCl₂ 5 mM; ZnCl₂ 0.1 mM; NaCl 0.3 M; but without AHA, and having a concentration of 80 μM. The sample is maintained at room temperature.

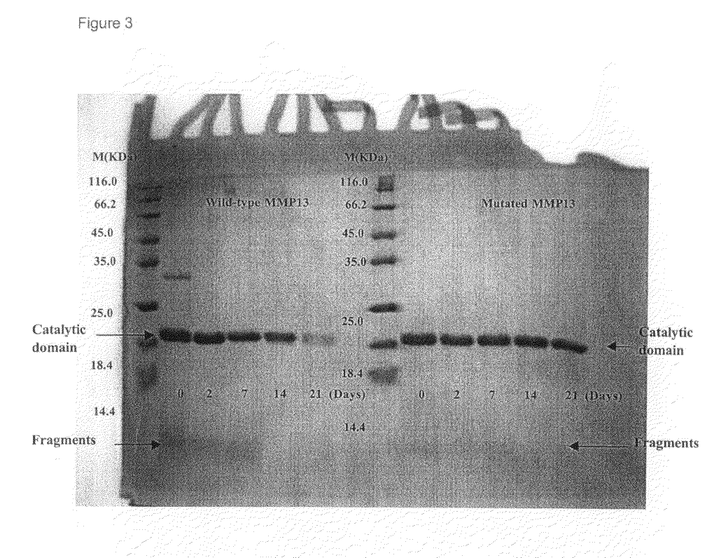

At intervals corresponding to 0, 2, 7, 14 and 21 days 5 μL aliquots of the two samples maintained at room temperature, are collected and immediately frozen at −80° C.; all the collected samples having the above said concentration are analysed by electrophoresis on SDS/polyacrylamide gel (17%), thus obtaining the results showed in FIG. 3 and illustrated in the following.

The analysis shows a significantly higher stability of the mutated protein in respect of the wild-type protein. In fact, it is evident from FIG. 3 a decrease of intensity of the band corresponding to the non degraded wild-type protein, which is almost disappeared after 21 days; on the contrary, the intensity of the same band for the protein mutated according to the invention, is almost unchanged after 21 days.

Moreover, FIG. 3 shows bands corresponding to low molecular weight fragments only for the wild-type protein, while no such bands are visible for the protein mutated according to the invention.

### Example 6

### Evaluation of the Activity

The activity measurements have been carried out with the following methodology. The colorimetric substrate is a thiopeptide (Ac-Pro-Leu-Gly-[2-mercapto-4-methyl-pentanoyl]-Leu-Gly-OC₂H₅, a reagent from Biomol catalogue. The hydrolysis of the substrate due to matrix metalloproteinases produces a sulphydrilic group that reacts with DTNB (5,5′-dithiobis(2-nitrobenzoic acid), called Ellman reagent) to produce 2-nitro-5-thiobenzoic acid, detectable using its absorbance at 412 nm. The reaction conditions are reported hereinafter:

Reaction cell=500 μl
[thiopeptolidel]*=100 μM
[protein]*=50 nM
*final concentration in the cell

Buffer for the activity measurement:

### 50 mM HEPES pH 7

### 5 mM CaCl₂

### 0.1 mM ZnCl₂

### 0.05% Brj-35

1 mM DTNB [5,5′-dithiobis(2-nitrobenzoic acid)]

The results at 25° are:

MMP3 (SEQ ID No. 3) (F171)—specific activity at 25° C.: 43 U/μg. An U=100 pmol/min
MMP7 (SEQ ID No. 4) (S166D)—specific activity at 25° C.: 66 U/μg. An U=100 pmol/min
MMP10 (SEQ ID No. 7) (F171)—specific activity at 25° C.: 41 U/μg. An U=100 pmol/min
MMP12 (SEQ ID No. 9) (F171)—specific activity at 25° C.: 91 U/μg. An U=100 pmol/min

The activity values of the MMP refer by Biomol are measured at 37° C. on the same substrate used by us, and they are as follows:

MMP3 (SEQ ID No. 3)—specific activity at 37° C.: 50 U/μg. An U=100 pmol/min
MMP7 (SEQ ID No. 4)—specific activity at 37° C.: 34.5 U/μg. An U=100 pmol/min
MMP10 (SEQ ID No. 7)—specific activity at 37° C.: 42.5 U/μg. An U=100 pmol/min
MMP12 (SEQ ID No. 9)—specific activity at 37° C.: 86 U/μg. An U=100 pmol/min

### Example 7

The cDNA of the catalytic domain for each MMP has been cloned in a pET21 vector (Novagen); the resulting vector has been then used for transformation of BL21-DE3 cells of Escherichia coli. The transformed cells have been grown in Luria-Bertani medium at a temperature of 37° C. The expression of the protein has been induced during the exponential growth phase by addition of IPTG 0.5 mM. 5 hours after induction, cells have been harvested and lysated, and inclusion bodies have been dissolved in a buffer containing Tris 20 mM pH 8 and urea 6 M. The proteins are then purified by means of an anionic exchange column (HiTrap QHP-Pharmacia) using a buffer containing Tris 20 mM pH 8 and urea 6 M, and eluting with a linear gradient of NaCl until 0.5 M.

The purified proteins have been then refolded by subsequent dialysis with solutions containing Tris 50 mM pH 7.2; CaCl₂ 10 mM; ZnCl₂ 0.1 M; NaCl 0.3 M and AHA 0.2 M. In the final refolding dialysis the concentration of AHA is brought to 0.5 M. For the isolation of the catalytic domains a chromatographic column Superdex 75 16/60 (Pharmacia), eluted with the buffer of the last dialysis, has been used.

Following the procedure described above, the catalytic domain of MMP-1 (SEQ ID No. 1), MMP-3 (SEQ ID No. 3), MMP-7 (SEQ ID No. 4), MMP-8 (SEQ ID No. 5), MMP-10 (SEQ ID No. 7) and MMP-12 (SEQ ID No. 9) have been prepared and purified.

1. A catalytic domain of human matrix metalloproteinases, wherein said catalytic domain is mutated so that the aminoacidic residue corresponding to phenylalanine 171 according to the numbering of the sequence Accession No. P39900 (SwissProt), is an hydrophilic and/or charged aminoacidic residue.

2. The catalytic domain according to claim 1, wherein said aminoacidic residue corresponding to phenylalanine 171 is selected among the following:
for human MMP-2 (SEQ ID No. 2) and all its isoforms determined by the alternative “splicing”, the aminoacidic residue that is mutated is glycine 181 according to the numbering of the sequence Accession No. P08253 (SwissProt);
for human MMP-3 (SEQ ID No. 3) and all its isoforms determined by the alternative “splicing”, the aminoacidic residue that is mutated is phenylalanine 171 according to the numbering of the sequence Accession No. P08254 (SwissProt) or Q6GRF8 (TrEMBL);
for human MMP-7 (SEQ ID No. 4) and all its isoforms determined by the alternative “splicing”, the aminoacidic residue that is mutated is serine 166 according to the numbering of the sequence Accession No. P09237 (SwissProt);
for human MMP-9 (SEQ ID No. 6) and all its isoforms determined by the alternative “splicing”, the aminoacidic residue that is mutated is glycine 178 according to the numbering of the sequence Accession No. P14780 (SwissProt);
for human MMP-10 (SEQ ID No. 7) and all its isoforms determined by the alternative “splicing”, the aminoacidic residue that is mutated is phenylalanine 170 according to the numbering of the sequence Accession No. P09238 (SwissProt) or Q53HH9 (TrEMBL);
for human MMP-12 (SEQ ID No. 9) and all its isoforms determined by the alternative “splicing”, the aminoacidic residue that is mutated is phenylalanine 171 according to the numbering of the sequence Accession No. P39900 (SwissProt);
for human MMP-13 (SEQ ID No. 10) and all its isoforms determined by the alternative “splicing”, the aminoacidic residue that is mutated is phenylalanine 175 according to the numbering of the sequence Accession No. P45452 (SwissProt) or Q7Z5M0, Q7Z5M1 and Q6WN6 (TrEMBL);
for human MMP-14 (SEQ ID No. 11) and all its isoforms determined by the alternative “splicing”, the aminoacidic residue that is mutated is serine 189 according to the numbering of the sequence Accession No. P50281 (SwissProt) and Q6GSF3 (TrEMBL);
for human MMP-15 (SEQ ID No. 12) and all its isoforms determined by the alternative “splicing”, the aminoacidic residue that is mutated is serine 209 according to the numbering of the sequence Accession No. P51511 (SwissProt) and Q7KZY0 (TrEMBL);
for human MMP-16 and all its isoforms (SEQ ID No. 13 and SEQ ID No. 14) determined by the alternative “splicing”, the aminoacidic residue that is mutated is serine 196 according to the numbering of the sequence Accession No. P51512 (SwissProt), or Q52H48 and Q14824 (TrEMBL);
for human MMP-17 (SEQ ID No. 15) and all its isoforms determined by the alternative “splicing”, the aminoacidic residue that is mutated is glycine 200 according to the numbering of the sequence Accession No. Q9ULZ9 (SwissProt), Q5U5M0 and Q81WC3 (TrEMBL);
for human MMP-19 and all its isoforms (SEQ ID No. 16 and SEQ ID No. 17) determined by the alternative “splicing”, the aminoacidic residue that is mutated is tyrosine 165 according to the numbering of the sequence Accession No. Q99542 (TrEMBL);
for human MMP-20 (SEQ ID No. 18) and all its isoforms determined by the alternative “splicing”, the aminoacidic residue that is mutated is serine 179 according to the numbering of the sequence Accession No. O60882 (TrEMBL);
for human MMP-21 (SEQ ID No. 19) and all its isoforms determined by the alternative “splicing”, the aminoacidic residue that is mutated is cysteine 238 according to the numbering of the sequence Accession No. Q5VZP9 and Q8N119 (TrEMBL);
for human MMP-23A (SEQ ID No. 20) and all its isoforms determined by the alternative “splicing”, the aminoacidic residue that is mutated is cysteine 152 according to the numbering of the sequence Accession No. O75900 (TrEMBL);
for human MMP-23B (SEQ ID No. 21) and all its isoforms determined by the alternative “splicing”, the aminoacidic residue that is mutated is cysteine 152 according to the numbering of the sequence Accession No. Q9UBR9 (TrEMBL);
for human MMP-24 (SEQ ID No. 22) and all its isoforms determined by the alternative “splicing”, the aminoacidic residue that is mutated is serine 232 according to the numbering of the sequence Accession No. Q9Y5R2 and Q9H440 (TrEMBL);
for human MMP-25 (SEQ ID No. 23) and all its isoforms determined by the alternative “splicing”, the aminoacidic residue that is mutated is serine 182 according to the numbering of the sequence Accession No. Q9NPA2 (TrEMBL);
for human MMP-26 (SEQ ID No. 24) and all its isoforms determined by the alternative “splicing”, the aminoacidic residue that is mutated is glycine 161 on the basis of the numbering of the sequence Accession No. Q9NRE1 (TrEMBL);
for human MMP-27 (SEQ ID No. 25) and all its isoforms determined by the alternative “splicing”, the aminoacidic residue that is mutated is cysteine 168 according to the numbering of the sequence Accession No. Q9H306 and Q6UWK6 (TrEMBL);
for human MMP-28 and all its isoforms (SEQ ID No. 26 and SEQ ID No. 27) determined by the alternative “splicing”, the aminoacidic residue that is mutated is glycine 193 according to the numbering of the sequence Accession No. Q9H239 and Q9BUG8 (TrEMBL);
for human MMP-like 1 (SEQ ID No. 28) and all its isoforms determined by the alternative “splicing”, the aminoacidic residue that is mutated is serine 106 according to the numbering of the sequence Accession No. O43923 (TrEMBL).

3. The catalytic domain according to claim 1, wherein said hydrophilic and/or charged aminoacidic residue is a residue of an aminoacid selected from the group consisting of glutamine, glutamic acid, asparagine and aspartic acid.

4. The catalytic domain according to claim 3, wherein said hydrophilic and/or charged aminoacidic residue is a residue of aspartic acid.

5. A mutated human matrix metalloproteinases comprising as catalytic domain the catalytic domain as defined in claim 1.

6. Use of human matrix metalloproteinases and of the catalytic domains thereof, optionally mutated as defined in claim 1, for the preparation of pharmaceutical compositions.

7. The use according to claim 6, for the preparation of pharmaceutical compositions useful for the treatment of pathologies to which an accumulation of matrix biopolymers and/or an excess of TIMPs (Tissutal Inhibitors of matrix MetalloProteinases) is associated.

8. The use according to claim 7, wherein said pathologies are selected among scleroderma, cardiosclerosis, nephrosclerosis, hepatic fibrosis, pulmonary fibrosis and pancreatic fibrosis.

9. Pharmaceutical compositions comprising as active principle the human matrix metalloproteinases or the catalytic domains thereof, optionally mutated as defined in claim 1.

10. A diagnostic kit for the diagnosis of pathologies to which accumulation of matrix biopolymers and/or an excess of TIMPs (Tissutal Inhibitors of matrix MetalloProteinases) is associated, comprising the human matrix metalloproteinases or the catalytic domains thereof, optionally mutated as defined in claim 1.

11. The diagnostic kit according to claim 10, wherein said pathologies are selected among scleroderma, cardiosclerosis, nephrosclerosis, hepatic fibrosis, pulmonary fibrosis and pancreatic fibrosis.

12. Use of mutated matrix metalloproteinases as defined in claim 5, or of the mutated catalytic domain of matrix metalloproteinases as defined in claim 1, as reagents for the pharmacological characterisation of the matrix metalloproteinases as pharmaceutical target.

13. A DNA sequence codifying the mutated matrix metalloproteinases as defined in claim 5.

14. A recombinant vector containing the DNA sequence as defined in claim 13.

15. An isolated cell transfected or transformed with the recombinant vector as defined in claim 14.


SEQUENCE LISTING (28 sequences)

SEQ ID NO: 1 - protein, 469 aa, Artificial Sequence
  Mutated Matrix Metalloproteinase-1
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 2 - protein, 660 aa, Artificial Sequence
  Mutated Matrix Metalloproteinase-2
MEALMARGALTGPLRALCLLGCLLSHAAAAPSPIIKFPGDVAPKTDKELAVQYLNTFYGCPKESCNLFVLKDTLKKMQKFFGLPQTGDLDQNTIETMRKPRCGNPDVANYNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 3 - protein, 477 aa, Artificial Sequence
  Mutated Matrix Metalloproteinase-3
MKSLPILLLLCVAVCSAYPLDGAARGEDTSMNLVQKYLENYYDLKKDVKQFVRRKDSGPVVKKIREMQKFLGLEVTGKLDSDTLEVMRKPRCGVPDVGHFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 4 - protein, 267 aa, Artificial Sequence
  Mutated Matrix Metalloproteinase-7
MRLTVLCAVCLLPGSLALPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 5 - protein, 467 aa, Artificial Sequence [sequence omitted]

SEQ ID NO: 6 - protein, 707 aa, Artificial Sequence
  Mutated Matrix Metalloproteinase-9
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 7 - protein, 476 aa, Artificial Sequence
  Mutated Matrix Metalloproteinase-10
MMHLAFLVLLCLPVCSAYPLSGAAKEEDSNKDLAQQYLEKYYNLEKDVKQFRRKDSNLIVKKIQGMQKFLGLEVTGKLDTDTLEVMRKPRCGVPDVGHFSSFPGMPKWRKTHLTYRIVNYTPDLPRDAVDSAIEKALKVWEEVTPLTFSRLYEGEADIMISFAVKEHGDFYSFDGPGHSLAHAYPPGPGLYGDIHFDDDEKWTEDASGTNLFLVAAHELGHSLGLFHSANTEALMYPLYNSFTELAQFRLSQDDVNGIQSLYGPPPASTEEPLVPTKSVPSGSEMPAKCDPALSFDAISTLRGEYLFFKDRYFWRRSHWNPEPEFHLISAFWPSLPSYLDAAYEVNSRDTVFIFKGNEFWAIRGNEVQAGYPRGIHTLGFPPTIRKIDAAVSDKEKKKTYFFAADKYWRFDENSQSMEQGFPRLIADDFPGVEPKVDAVLQAFGFFYFFSGSSQFEFDPNARMVTHILKSNSWLHC

SEQ ID NO: 8 - protein, 488 aa, Artificial Sequence [sequence omitted]

SEQ ID NO: 9 - protein, 470 aa, Artificial Sequence
  Mutated Matrix Metalloproteinase-12
MKFLLILLLQATASGALPLNSSTSLEKNNVLFGERYLEKFYGLEINKLPVTKMKYSGNLMKEKIQEMQHFLGLKVTGQLDTSTLEMMHAPRCGVPDVHHFREMPGGPVWRKHYITYRINNYTPDMNREDVDYAIRKAFQVWSNVTPLKFSKINTGMADILVVFARGAHGDFHAFDGKGGILAHAFGPGSGIGGDAHFDEDEFWTTHSGGTNLFLTAVHEIGHSLGLGHSSDPKAVMFPTYKYVDINTFRLSADDIRGIQSLYGDPKENQRLPNPDNSEPALCDPNLSFDAVTTVGNKIFFFKDRFFWLKVSERPKTSVNLISSLWPTLPSGIEAAYEIEARNQVFLFKDDKYWLISNLRPEPNYPKSIHSFGFPNFVKKIDAAVFNPRFYRTYFFVDNQYWRYDERRQMMDPGYPKLITKNFQGIGPKIDAVFYSKNKYYYFFQGSNQFEYDFLLQRITKTLKSNSWFGC

SEQ ID NO: 10 - protein, 471 aa, Artificial Sequence
  Mutated Matrix Metalloproteinase-13
MHPGVLAAFLFLSWTHCRALPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGPGDEDPNPKHPKTPDKCDPSLSLDAITSLRGETMIFKDRFFWRLHPQQVDAELFLTKSFWPELPNRIDAAYEHPSHDLIFIFRGRKFWALNGYDILEGYPKKISELGLPKEVKKISAAVHFEDTGKTLLFSGNQVWRYDDTNHIMDKDYPRLIEEDFPGIGDKVDAVYEKNGYIYFFNGPIQFEYSIWSNRIVRVMPANSILWC

SEQ ID NO: 11 - protein, 582 aa, Artificial Sequence
  Mutated Matrix Metalloproteinase-14
MSPAPRPSRCLLLPLLTLGTALASLGSAQSSSFSPEAWLQQYGYLPPGDLRTHTQRSPQSLSAAIAAMQKFYGLQVTGKADADTMKAMRRPRCGVPDKFGAEIKANVRRKRYAIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSGGRPDEGTEEETEVIIIEVDEEGGGAVSAAAVVLPVLLLLLVLAVGLAVFFFRRHGTPRRLLYCQRSLLDKV

SEQ ID NO: 12 - protein, 669 aa, Artificial Sequence
  Mutated Matrix Metalloproteinase-15
MGSDPSAPGRPGWTGSLLGDREEAARPRLLPLLLVLLGCLGLGVAAEDAEVHAENWLRLYGYLPQPSRHMSTMRSAQILASALAEMQRFYGIPVTGVLDEETKEWMKRPRCGVPDQFGVRVKANLRRRRKRYALTGRKWNNHHLTFSIQNYTEKLGWYHSMEAVRRAFRVWEQATPLVFQEVPYEDIRLRRQKEADIMVLFASGFHGDSSPFDGTGGFLAHAYFPGPGLGGDTHFDADEPWTFSSTDLHGNNLFLVAVHELGHALGLEHSSNPNAIMAPFYQWKDVDNFKLPEDDLRGIQQLYGTPDGQPQPTQPLPTVTPRRPGRPDHRPPRPPQPPPPGGKPERPPKPGPPVQPRATERPDQYGPNICDGDFDTVAMLRGEMFVFKGRWFWRVRHNRVLDNYPMPIGHFWRGLPGDISAAYERQDGRFVFFKGDRYWLFREANLEPGYPQPLTSYGLGIPYDRIDTAIWWEPTGHTFFFQEDRYWRFNEETQRGDPGYPKPISVWQGIPASPKGAFLSNDAAYTYFYKGTKYWKFDNERLRMEPGYPKSILRDFMGCQEHVEPGPRWPDVARPPFNPHGGAEPGADSAEGDVGDGDGDFGAGVNKDGGSRVVVQMEEVARTVNVVMVLVPLLLLLCVLGLTYALVQMQRKGAPRVLLYCKRSLQEWV

SEQ ID NO: 13 - protein, 607 aa, Artificial Sequence
  Mutated Matrix Metalloproteinase-16 Isoform 1
MILLTFSTGRRLDFVHHSGVFFLQTLLWILCATVCGTEQYFNVEVWLQKYGYLPPTDPRMSVLRSAETMQSALAAMQQFYGINMTGKVDRNTIDWMKKPRCGVPDQTRGSSKFHIRRKRYALTGQKWQHKHITYSIKNVTPKVGDPETRKAIRRAFDVWQNVTPLTFEEVPYSELENGKRDVDITIIFASGFHGDSSPFDGEGGFLAHAYFPGPGIGGDTHFDSDEPWTLGNPNHDGNDLFLVAVHELGHALGLEHSNDPTAIMAPFYQYMETDNFKLPNDDLQGIQKIYGPPDKIPPPTRPLPTVPPHRSIPPADPRKNDRPKPPRPPTGRPSYPGAKPNICDGNFNTLAILRREMFVFKDQWFWRVRNNRVMDGYPMQITYFWRGLPPSIDAVYENSDGNFVFFKGNKYWVFKDTTLQPGYPHDLITLGSGIPPHGIDSAIWWEDVGKTYFFKGDRYWRYSEEMKTMDPGYPKPITVWKGIPESPQGAFVHKENGFTYFYKGKEYWKFNNQILKVEPGYPRSILKDFMGCDGPTDRVKEGHSPPDDVDIVIKLDNTASTVKAIAIVIPCILALCLLVLVYTVFQFKRKGTPRHILYCKRSMQEWV

SEQ ID NO: 14 - protein, 457 aa, Artificial Sequence
  Mutated Matrix Metalloproteinase-16 Isoform 2
MILLTFSTGRRLDFVHHSGVFFLQTLLWILCATVCGTEQYFNVEVWLQKYGYLPPTDPRMSVLRSAETMQSALAAMQQFYGINMTGKVDRNTIDWMKKPRCGVPDQTRGSSKFHIRRKRYALTGQKWQHKHITYSIKNVTPKVGDPETRKAIRRAFDVWQNVTPLTFEEVPYSELENGKRDVDITIIFASGFHGDSSPFDGEGGFLAHAYFPGPGIGGDTHFDSDEPWTLGNPNHDGNDLFLVAVHELGHALGLEHSNDPTAIMAPFYQYMETDNFKLPNDDLQGIQKIYGPPDKIPPPTRPLPTVPPHRSIPPADPRKNDRPKPPRPPTGRPSYPGAKPNICDGNFNTLAILRREMFVFKDQWFWRVRNNRVMDGYPMQITYFWRGLPPSIDAVYENSDGNFVFFKVKGDTLSVIQDGWLYKYHWKWILEQRQSVPVLSRQTEKHKTYEELSSITY

SEQ ID NO: 15 - protein, 606 aa, Artificial Sequence
  Mutated Matrix Metalloproteinase-17
MRRRAARGPGPPPPGPGLSRLPLLPLPLLLLLALGTRGGCAAPAPAPRAEDLSLGVEWLSRFGYLPPADPTTGQLQTQEELSKAITAMQQFGGLEATGILDEATLALMKTPRCSLPDLPVLTQARRRRQAPAPTKWNKRNLSWRVRTFPRDSPLGHDTVRALMYYALKVWSDIAPLNFHEVAGSTADIQIDFSKADHNDGYPFDGPGGTVAHAFFPGHHHTAGDTHFDDDEAWTFRSSDAHGMDLFAVAVHEFGHAIGLSHVAAAHSIMRPYYQGPVGDPLRYGLPYEDKVRVWQLYGVRESVSPTAQPEEPPLLPEPPDNRSSAPPRKDVPHRCSTHFDAVAQIRGEAFFFKGKYFWRLTRDRHLVSLQPAQMHRFWRGLPLHLDSVDAVYERTSDHKIVFFKGDRYWVFKDNNVEEGYPRPVSDFSLPPGGIDAAFSWAHNDRTYFFKDQLYWRYDDHTRHMDPGYPAQSPLWRGVPSTLDDAMRWSDGASYFFRGQEYWKVLDGELEVAPGYPQSTARDWLVCGDSQADGSVAAGVDAAEGPRAPPGQHDQSRSEDGYEVCSCTSGASSPPGAPGPLVAATMLLLLPPLSPGALWTAAQALTL

SEQ ID NO: 16 - protein, 508 aa, Artificial Sequence
  Mutated Matrix Metalloproteinase-19 Isoform rasi-1
MNCQQLWLGFLLPMTVSGRVLGLAEVAPVDYLSQYGYLQKPLEGSNNFKPEDITEALRAFQEASELPVSGQLDDATRARMRQPRCGLEDPFNQKTLKYLLLGRWRKKHLTFRILNLPSTLPPHTARAALRQAFQDWSNVAPLTFQEVQAGAADIRLSFHGRQSSYCSNTFDGPGRVLAHADIPELGSVHFDEDEFWTEGTYRGVNLRIIAAHEVGHALGLGHSRYSQALMAPVYEGYRPHFKLHPDDVAGIQALYGKKSPVIRDEEEEETELPTVPPVPTEPSPMPDPCSSELDAMMLGPRGKTYAFKGDYVWTVSDSGPGPLFRVSALWEGLPGNLDAAVYSPRTQWIHFFKGDKVWRYINFKMSPGFPKKLNRVEPNLDAALYWPLNQKVFLFKGSGYWQWDELARTDFSSYPKPIKGLFTGVPNQPSAAMSWQDGRVYFFKGKVYWRLNQQLRVEKGYPRNISHNWMHCRPRTIDTTPSGGNTTPSGTGITLDTTLSATETTFEY

SEQ ID NO: 17 - protein, 308 aa, Artificial Sequence
  Mutated Matrix Metalloproteinase-19 Isoform rasi-6
MRQPRCGLEDPFNQKTLKYLLLGRWRKKHLTFRILNLPSTLPPHTARAALRQAFQDWSNVAPLTFQEVQAGAADIRLSFHGRQSSYCSNTFDGPGRVLAHADIPELGSVHFDEDEFWTEGTYRGVNLRIIAAHEVGHALGLGHSRYSQALMAPVYEGYRPHFKLHPDDVAGIQALYGKKSPVIRDEEEEETELPTVPPVPTEPSPMPDPCSSELDAMMLGEAPPLQAVGRRWGQPADPEAWTNGSDMGLQHEQWRAPWEDLCFQGGLCVDCIRFRTGPLVPSVCPLGGAPRKPGCCCLLASNTMDSLL

SEQ ID NO: 18 - protein, 483 aa, Artificial Sequence [sequence omitted]

SEQ ID NO: 19 - protein, 569 aa, Artificial Sequence [sequence omitted]

SEQ ID NO: 20 - protein, 390 aa, Artificial Sequence [sequence omitted]

SEQ ID NO: 21 - protein, 390 aa, Artificial Sequence
  Mutated Matrix Metalloproteinase-23B
MGRGARVPSEAPGAGVERRWLGAALVALCLLPALVLLARLGAPAVPAWSAAQGDVAALGLSAVPPTRVPGPLAPRRRRYTLTPARLRWDHFNLTYRILSFPRNLLSPRETRRALAAAFRMWSDVSPFSFREVAPEQPSDLRIGFYPINHTDCLVSALHHCFDGPTGELAHAFFPPHGGIHFDDSEYWVLGPTRYSWKKGVWLTDLVHVAAHEIGHALGLMHSQHGRALMHLNATLRGWKALSQDELWGLHRLYGCLDRLFVCASWARRGFCDARRRLMKRLCPSSCDFCYEFPFPTVATTPPPPRTKTRLVPEGRNVTFRCGQKILHKKGKVYWYKDQEPLEFSYPGYLALGEAHLSIIANAVNEGTYTCVVRRQQRVLTTYSWRVRVRG

SEQ ID NO: 22 - protein, 645 aa, Artificial Sequence
  Mutated Matrix Metalloproteinase-24
MPRSRGGRAAPGPPPPPPPPGQAPRWSRWRVPGRLLLLLLPALCCLPGAARAAAAAAGAGNRAAVAVAVARADEAEAPFAGQNWLKSYGYLLPYDSRASALHSAKALQSAVSTMQQFYGIPVTGVLDQTTIEWMKKPRCGVPDHPHLSRRRRNKRYALTGQKWRQKHITYSIHNYTPKVGELDTRKAIRQAFDVWQKVTPLTFEEVPYHEIKSDRKEADIMIFFASGFHGDSSPFDGEGGFLAHAYFPGPGIGGDTHFDSDEPWTLGNANHDGNDLFLVAVHELGHALGLEHSSDPSAIMAPFYQYMETHNFKLPQDDLQGIQKIYGPPAEPLEPTRPLPTLPVRRIHSPSERKHERQPRPPRPPLGDRPSTPGTKPNICDGNFNTVALFRGEMFVFKDRWFWRLRNNRVQEGYPMQIEQFWKGLPARIDAAYERADGRFVFFKGDKYWVFKEVTVEPGYPHSLGELGSCLPREGIDTALRWEPVGKTYFFKGERYWRYSEERRATDPGYPKPITVWKGIPQAPQGAFISKEGYYTYFYKGRDYWKFDNQKLSVEPGYPRNILRDWMGCNQKEVERRKERRLPQDDVDIMVTINDVPGSVNAVAVVIPCILSLCILVLVYTIFQFKNKTGPQPVTYYKRPVQEWV

SEQ ID NO: 23 - protein, 562 aa, Artificial Sequence
  Mutated Matrix Metalloproteinase-25
MRLRLRLLALLLLLLAPPARAPKPSAQDVSLGVDWLTRYGYLPPPHPAQAQLQSPEKLRDAIKVMQRFAGLPETGRMDPGTVATMRKPRCSLPDVLGVAGLVRRRRRYALSGSVWKKRTLTWRVRSFPQSSQLSQETVRVLMSYALMAWGMESGLTFHEVDSPQGQEPDILIDFARAFHQDSYPFDGLGGTLAHAFFPGEHPISGDTHFDDEETWTFGSKDGEGTDLFAVAVHEFGHALGLGHSSAPNSIMRPFYQGPVGDPDKYRLSQDDRDGLQQLYGKAPQTPYDKPTRKPLAPPPQPPASPTHSPSFPIPDRCEGNFDAIANIRGETFFFKGPWFWRLQPSGQLVSPRPARLHRFWEGLPAQVRVVQAAYARHRDGRILLFSGPQFWVFQDRQLEGGARPLTELGLPPGEEVDAVFSWPQNGKTYLVRGRQYWRYDEAAARPDPGYPRDLSLWEGAPPSPDDVTVSNAGDTYFFKGAHYWRFPKNSIKTEPDAPQPMGPNWLDCPAPSSGPRAPRPPKATPVSETCDCQCELNQAAGRWPAPIPLLLLPLLVGGVASR

SEQ ID NO: 24 - protein, 261 aa, Artificial Sequence
  Mutated Matrix Metalloproteinase-26
MQLVILRVTIFLPWCFAVPVPPAADHKGWDFVEGYFHQFFLTKKESPLLTQETQTQLLQQFHRNGTDLLDMQMHALLHQPHCGVPDGSDTSISPGRCKWNKHTLTYRIINYPHDMKPSAVKDSIYNAVSIWSNVTPLIFQQVQNGDADIKVSFWQWAHEDGWPFDGPGGILGHAFLPNSGNPGVVHFDKNEHWSASDTGYNLFLVATHEIGHSLGLQHSGNQSSIMYPTYWYHDPRTFQLSADDIQRIQHLYGEKCSSDIP

SEQ ID NO: 25 - protein, 513 aa, Artificial Sequence
  Mutated Matrix Metalloproteinase-27
MKRLLLLFLFFITFSSAFPLVRMMENEENVQLAQAYLNQFYSLEIEGNHLVQSKNRSLIDDKIREMQAFFGLTVTGKLDSNTLEIMKTPRCGVPDVGQYGYTLPGWRKYNLTYRIINYTPDMARAAVDEAIQEGLEVWSKVTPLKFTKISKGIADIMIAFRTRVHGRCPRYFDGPLGVLGHAFPPGPGLGGDTHFDEDENWTKDGAGFNLFLVAAHEFGHALGLSHSNDQTALMFPNYVSLDPRKYPLSQDDINGIQSIYGGLPKEPAKPKEPTIPHACDPDLTFDAITTFRREVMFFKGRHLWRIYYDITDVEFELIASFWPSLPADLQAAYENPRDKILVFKDENFWMIRGYAVLPDYPKSIHTLGFPGRVKKIDAAVCDKTTRKTYFFVGIWCWRFDEMTQTMDKGFPQRVVKHFPGISIRVDAAFQYKGFFFFSRGSKQFEYDIKTKNITRIMRTNTWFQCKEPKNSSFGFDINKEKAHSGGIKILYHKSLSLFIFGIVHLLKNTSIYQ

SEQ ID NO: 26 - protein, 520 aa, Artificial Sequence
  Mutated Matrix Metalloproteinase-28 Isoform 1
MVARVGLLLRALQLLLWGHLDAQPAERGGQELRKEAEAFLEKYGYLNEQVPKAPTSTRFSDAIRAFQWVSQLPVSGVLDRATLRQMTRPRCGVTDTNSYAAWAERISDLFARHRTKMRRKKRFAKQGNKWYKQHLSYRLVNWPEHLPEPAVRGAVRAAFQLWSNVSALEFWEAPATGPADIRLTFFQGDHNDGLGNAFDGPGGALAHAFLPRRGEAHFDQDERWSLSRRRGRNLFVVLAHEIGHTLGLTHSPAPRALMAPYYKRLGRDALLSWDDVLAVQSLYGKPLGGSVAVQLPGKLFTDFETWDSYSPQGRRPETQGPKYCHSSFDAITVDRQQQLYIFKGSHFWEVAADGNVSEPRPLQERWVGLPPNIEAAAVSLNDGDFYFFKGGRCWRFRGPKPVWGLPQLCRAGGLPRHPDAALFFPPLRRLILFKGARYYVLARGGLQVEPYYPRSLQDWGGIPEEVSGALPRPDGSIIFFRDDRYWRLDQAKLQATTSGRWATELPWMGCWHANSGSALF

SEQ ID NO: 27 - protein, 393 aa, Artificial Sequence
  Mutated Matrix Metalloproteinase-28 Isoform 2
MVARVGLLLRALQLLLWGHLDAQPAERGGQELRKEAEAFLEKYGYLNEQVPKAPTSTRFSDAIRAFQWVSQLPVSGVLDRATLRQMTRPRCGVTDTNSYAAWAERISDLFARHRTKMRRKKRFAKQGNKWYKQHLSYRLVNWPEHLPEPAVRGAVRAAFQLWSNVSALEFWEAPATGPADIRLTFFQGDHNDGLGNAFDGPGGALAHAFLPRRGEAHFDQDERWSLSRRRGRNLFVVLAHEIGHTLGLTHSPAPRALMAPYYKRLGRDALLSWDDVLAVQSLYGKPLGGSVAVQLPGKLFTDFETWDSYSPQGRRPETQGPKYCHSSFDAITVDRQQQLYIFKGSHFWEVAADGNVSEPRPLQERWVGLPPNIEAAAVSLNDGDFYFFKVQSV

SEQ ID NO: 28 - protein, 183 aa, Artificial Sequence
  Mutated Matrix Metalloproteinase-like 1
MDPGTVATMRKPRCSLPDVLGVAGLVRRRRRYALSGSVWKKRTLTWRVRSFPQSSQLSQETVRVLMSYALMAWGMESGLTFHEVDSPQGQEPDILIDFARAFHQDSYPFDGLGGTLAHAFFPGEHPISGDTHFDDEETWTFGSKASQQLEQELAGGSPVDEELGFSRGWRVNPLGPGSPERLS